(19) United States
(12) Patent Application Publication
(10) Pub. No.: US 2020/0330004 A1
(43) Pub. Date: Oct. 22, 2020

(54) METHODS AND DEVICES FOR IN SITU DETECTION OF A COMPOSITION OF A FLUID WITHIN A GASTROINTESTINAL TRACT

(71) Applicants: Khan Arif Wahid, Saskatoon (CA); Alimul Haque Khan, Saskatoon (CA)

(72) Inventors: Khan Arif Wahid, Saskatoon (CA); Alimul Haque Khan, Saskatoon (CA)

(21) Appl. No.: 16/849,124

(22) Filed: Apr. 15, 2020

Related U.S. Application Data
(60) Provisional application No. 62/835,220, filed on Apr. 17, 2019.

(51) Int. Cl. A61B 5/1459; A61B 5/00; A61B 5/02

(52) U.S. Cl. CPC A61B 5/1459; A61B 5/42; A61B 5/6861; A61B 5/02042; A61B 5/073; A61B 5/7282; A61B 2560/0475; A61B 2562/0233; A61B 5/7246

(57) ABSTRACT

Methods and devices for in situ detection of a target substance within a fluid within a gastrointestinal tract are provided. The methods and devices use optical properties such as reflection or transmission of light to distinguish blood solutions from non-blood solutions in a gastrointestinal tract.

### CROSS-REFERENCE TO RELATED APPLICATION

This application claims the benefit of U.S. Provisional Patent Application No. 62/835,220, filed Apr. 17, 2019, the entire content of which is incorporated herein by reference.

### FIELD

The described embodiments relate to methods and devices for detecting a composition of a fluid and, in particular, to in situ detection of a target substance in a fluid, such as blood, within a gastrointestinal tract using an ingestible wireless capsule device.

### INTRODUCTION

Gastrointestinal (GI) bleeding in humans and animals is not uncommon, and may sometimes lead to fatal consequences. Types of GI bleeding may include acute and chronic bleeding, as well as upper and lower GI bleeding.

One technique for detecting GI bleeding is through wireless capsule endoscopy. Patients may swallow an electronic capsule-shaped device which captures thousands of images in the GI tract. The images may be stored locally at the device for later retrieval and analysis, or may be sent from the capsule device to a remote workstation via wireless communication. The images may be analyzed at the workstation by a clinician in order to detect bleeding, as well as other GI abnormalities.

The use of images to detect GI bleeding can be a time-consuming and error-prone process. In particular, clinicians can sometimes fail to visually identify the bleeding in the received images.

### SUMMARY

In a broad aspect, there is provided a method of in situ detection of a composition of a fluid within a gastrointestinal tract using an ingestible device, the method comprising: transmitting, using at least one light transmission source of the ingestible device, at least one probe signal from the at least one light transmission source, the at least one probe signal comprising at least two wavelengths of light with respective intensities; detecting, using at least one optical sensor of the ingestible device, the at least one probe signal following at least one interaction of the at least one probe signal with the fluid to generate a received signal; comparing the respective intensities of the at least two wavelengths of light within the received signal; based on the comparing, determining whether the fluid contains a target substance; and updating a memory of the ingestible device with the determination.

In some cases, the interaction of the at least one probe signal with the fluid comprises a reflection from the fluid, and wherein the comparing comprises comparing a ratio of respective reflection intensities of the at least two wavelengths of light from the fluid.

In some cases, the first wavelength is substantially about 700 nm and the second wavelength is substantially about 630 nm. In some cases, the first wavelength is substantially about 480 nm and the second wavelength is substantially about 530 nm.

In some cases, the determining comprises determining whether the ratio exceeds a predetermined reflectance threshold. In some cases, the predetermined reflectance threshold is substantially within a range of 1.2 to 1.3. In some cases, predetermined reflectance threshold is substantially within a range of 0.9 to 1.0.

In some cases, the interaction of the at least one probe signal with the fluid comprises a transmission through the fluid, and wherein the comparing comprises comparing respective transmission intensities of the at least two wavelengths of light through the fluid with a predetermined transmission threshold.

In some cases, the determining comprises analyzing the ratio of respective reflection intensities and the respective transmission intensities of the at least two wavelengths of light.

In some cases, the first wavelength corresponds to red light and the second wavelength corresponds to infrared light. In some cases, the first wavelength is substantially about 660 nm and the second wavelength is substantially about 880 nm. In some cases, the predetermined transmission threshold is substantially 101.

In some cases, the interaction of the at least one probe signal with the fluid comprises a reflection from the fluid, wherein the at least one probe signal comprises at least three wavelengths of light, and wherein the comparing comprises comparing respective reflectance intensities of the at least three wavelengths of light from the fluid.

In some cases, the determining comprises analyzing the ratio of respective reflection intensities and the respective reflectance intensities of the at least three wavelengths of light.

In some cases, the determining comprises analyzing the ratio of respective reflection intensities and the respective transmission intensities of the at least two wavelengths of light.

In some cases, the first wavelength corresponds to red light, the second wavelength corresponds to green light and the third wavelength corresponds to blue light.

In some cases, the comparing comprises comparing reflectance intensities of each of the at least three wavelengths of light to one or more predetermined reflectance thresholds.

In some cases, a first predetermined reflectance threshold of the one or more predetermined reflectance thresholds is determined according to the formula: Inb−x*Inr, where Inb is a reflectance intensity of the third wavelength, Inr is a reflectance intensity of the first wavelength, and x is a first comparison factor. In some cases, x is substantially 8%. In some cases, x is 0.0814.

In some cases, a second predetermined reflectance threshold of the one or more predetermined reflectance thresholds is determined according to the formula: Ing−y*Inr, where Ing is a reflectance intensity of the second wavelength, Inr is a reflectance intensity of the first wavelength, and y is a second comparison factor. In some cases, y is substantially 10%. In some cases, y is 0.1083.

In some cases, a third predetermined reflectance threshold of the one or more predetermined reflectance thresholds is determined according to the formula: Inb−z*Ing, where Inb is a reflectance intensity of the third wavelength, Ing is a reflectance intensity of the second wavelength, and z is a third comparison factor. In some cases, z is substantially 69%. In some cases, z is 0.6911.

In some cases, the target substance is blood.

In another broad aspect, there is provided a non-transitory computer readable medium storing one or more computer-executable instructions, which when executed by a processor, causes the processor to perform a method as described herein.

In still another broad aspect, there is provided an ingestible device comprising: an electronic circuit, the electronic circuit comprising a processor, a memory and an energy storage element; a device body with a first end and a second end opposed along a longitudinal axis; a first end portion of the device body positioned at the first end of the device body along the longitudinal axis, the first end portion having a recess therein, the recess defined by a first sidewall, a second sidewall and a base wall, the first and second sidewalls laterally spaced apart; at least one light transmission source coupled to the electronic circuit and positioned to emit light generally perpendicularly to the first sidewall; and at least one optical sensor coupled to the electronic circuit and positioned in at least one of the first sidewall and the second sidewall.

In some cases, the device further comprises: a second end portion of the device body positioned at the second end of the device body along the longitudinal axis; and an image sensor provided in the second end portion. In some cases, the second end portion comprises a substantially transparent cover.

### BRIEF DESCRIPTION OF THE DRAWINGS

A preferred embodiment of the present invention will now be described in detail with reference to the drawings, in which:

FIG. 1 is a simplified schematic diagram of a wireless capsule endoscopy system in accordance with at least some embodiments;

FIG. 2 is a simplified block diagram of an example wireless capsule;

FIG. 3A is a perspective view of a wireless capsule in accordance with some embodiments;

FIG. 3B is an enlarged perspective view of a first end portion of the wireless capsule of FIG. 3A;

FIG. 3C is an enlarged perspective view of a first end portion of the wireless capsule of FIG. 3A in another embodiment;

FIG. 3D is a perspective view of the device of FIG. 3A;

FIG. 3E is a rotated side view of the device of FIG. 3A;

FIG. 3F is another rotated side view of the device of FIG. 3A;

FIGS. 4A and 4B are process flow diagrams showing an example process for in situ detection of a composition of a fluid in a gastrointestinal tract, which may use the wireless capsule of FIG. 3A;

FIG. 5 is a process flow diagram showing an example detection process in accordance with some embodiments;

FIGS. 6A to 6D are plots illustrating data generated according to the process of FIG. 5;

FIG. 7 is a process flow diagram illustrating another example detection process in accordance with some embodiments;

FIGS. 8A and 8B are plots illustrating data generated according to the process of FIG. 7;

FIG. 9 is a process flow diagram illustrating still another example detection process in accordance with some embodiments; and

FIGS. 10A to 10D are plots illustrating data generated according to the process of FIG. 9.

### DESCRIPTION OF EXEMPLARY EMBODIMENTS

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details, or with other methods, components, materials, etc. In other instances, well-known methods, procedures and components have not been described in detail since these are known to those skilled in the art. Furthermore, it should be noted that this description is not intended to limit the scope of the embodiments described herein, but rather as merely describing one or more exemplary implementations.

Unless the context requires otherwise, throughout the specification and claims which follow, the word “comprise” and variations thereof, such as, “comprises” and “comprising” are to be construed in an open, inclusive sense, that is as “including, but not limited to.”

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

Reference throughout this specification to “one embodiment” or “an embodiment” means that a particular feature, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

The headings and Abstract of the Disclosure provided herein are for convenience only and do not interpret the scope or meaning of the embodiments.

The terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Exemplary communicative couplings include, but are not limited to, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

It should also be noted that there may be some elements that are used to implement at least part of one of the embodiments described herein that may be implemented via software that is written in a high-level computer programming language such as one that employs an object-oriented paradigm. Accordingly, the program code may be written in Java, C++ or any other suitable programming language and may comprise modules or classes, as is known to those skilled in object-oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, EEPROM, magnetic disk, optical disc) or a device that is readable by a general or special purpose programmable device. The software program code, when read by the programmable device, configures the programmable device to operate in a new, specific and predefined manner in order to perform at least one of the methods described herein.

The description sets forth various embodiments of the systems, devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs executed by one or more computers (e.g., as one or more programs running on one or more computer systems), as one or more programs executed by on one or more controllers (e.g., microcontrollers) as one or more programs executed by one or more processors (e.g., microprocessors, central processing units, graphical processing units), as firmware, or as virtually any combination thereof, and that designing the circuitry and/or writing the code for the software and or firmware would be well within the skill of one of ordinary skill in the art in light of the teachings of this disclosure.

When logic is implemented as software and stored in memory, logic or information can be stored on any processor-readable medium for use by or in connection with any processor-related system or method. In the context of this disclosure, a memory is a processor-readable medium that is an electronic, magnetic, optical, or other physical device or means that contains or stores a computer and/or processor program. Logic and/or the information can be embodied in any processor-readable medium for use by or in connection with an instruction execution system, apparatus, or device, such as a computer-based system, processor-containing system, or other system that can fetch the instructions from the instruction execution system, apparatus, or device and execute the instructions associated with logic and/or information.

In the context of this specification, a “non-transitory computer-readable medium” can be any element that can store the program associated with logic and/or information for use by or in connection with the instruction execution system, apparatus, and/or device. The processor-readable medium can be, for example, but is not limited to, an electronic, magnetic, optical, electromagnetic, infrared, or semiconductor system, apparatus or device. More specific examples (a non-exhaustive list) of the computer readable medium would include the following: a portable computer diskette (magnetic, compact flash card, secure digital, or the like), a random access memory (RAM), a read-only memory (ROM), an erasable programmable read-only memory (EPROM, EEPROM, or Flash memory), a portable compact disc read-only memory (CDROM), digital tape, and other non-transitory media.

A wireless capsule may be provided to conduct in situ detection of abnormalities in a GI tract using one or more sensors. The sensors may detect the interaction of light with fluids in the GI tract with a view to determining the presence of a substance, such as blood. The capsule may use optical properties unique to the target substance (e.g., blood) in order to differentiate target solutions from non-target solutions.

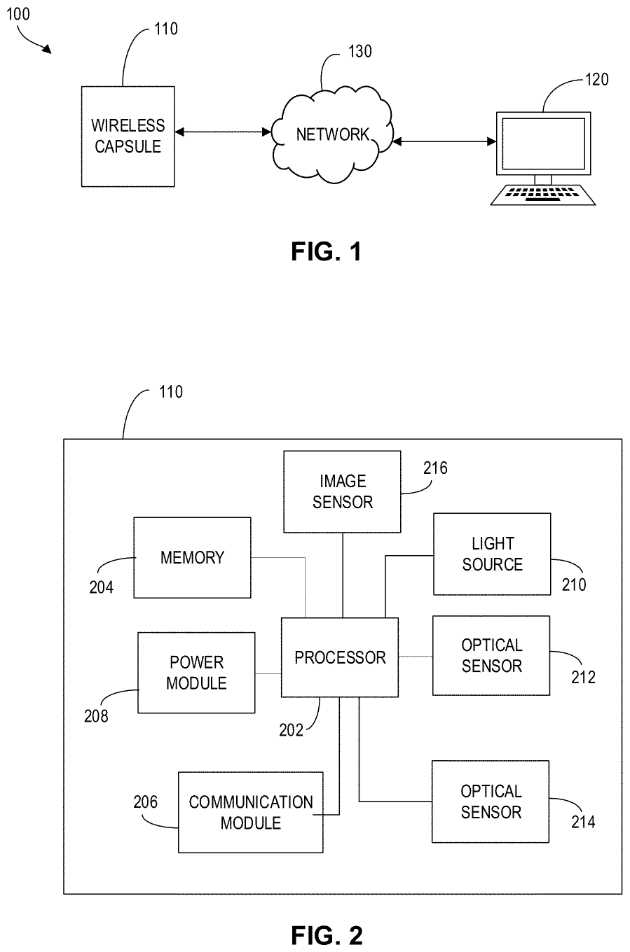

Referring now to FIG. 1, there is illustrated a schematic block diagram of a wireless endoscopy system 100. Wireless endoscopy system 100 provides the environment in which the devices and/or methods described herein generally operate. The system 100 generally has an ingestible device 110 in data communication with a remote terminal (or workstation) 120. The ingestible device 110 may communicate with remote terminal 120 through a network 130. Network 130 may be, for example, a wireless personal area network such as a Bluetooth™ network, a wireless local area network such as the IEEE 802.11 family of networks or, in some cases, a wired network or communication link such as a Universal Serial Bus (USB) interface or IEEE 802.3 (Ethernet) network, or others. In some embodiments, the ingestible device 110 may communicate with the remote terminal 120 in real-time, while in situ in a patient's gastrointestinal tract. In other embodiments, the ingestible device 110 may store images for later transmission once the capsule is retrieved from the patient.

Referring now to FIG. 2, there is illustrated a simplified block diagram of the ingestible device 110 in accordance with some embodiments. The ingestible device 110 may be, for example, an ingestible device adaptable to be swallowed by a user or patient and to pass through a patient's gastrointestinal tract. The ingestible device 110 generally has at least a processor 202 in communication with a memory 204, power module 208, communication module 110, at least one light source 210, at least a first optical sensor 212, a second optical sensor 214, and an image sensor 216.

Processor 202 may be configured to execute a plurality of instructions to control and operate the various components of the ingestible device 110. In some embodiments, the instructions may be transmitted from remote terminal 120 to the processor 202 using communication module 206. In other embodiments, the processor may be pre-configured with specific instructions. The pre-configured instructions may be executed in response to specific events or specific sequences of events, or at specific time intervals. Processor 202 may also be configured to receive information from the various components of ingestible device 110 and to make specific determinations using this information, as described further herein. The determinations may then be transmitted to the memory device 204 and/or the communication module 206.

Memory 204 may be, for example, a non-volatile read-write memory which stores computer-executable instructions and data, and a volatile memory (e.g., random access memory) that may be used as a working memory by processor 202.

The power module 208 may be, for example, a battery capable of supplying power to the ingestible device 110 for a predetermined period of time. In some other embodiments, power module 208 may be an inductive power module, which can receive wirelessly transmitted power and supply power to the ingestible device 110.

Communication module 206 may be configured to send and receive data, or information, to and from remote terminal 120. Communication module 206 may, for example, comprise a wireless transmitter or transceiver and antenna as described herein. In some embodiments, the communication module 206 may receive instructions or data from the remote terminal 120 and transmit the instructions or data to the processor 202. The communication module 206 may also transmit information and data, such as images or video, gathered by the ingestible device 110 to the remote terminal 120. Accordingly, communication module 206 can be configured to provide duplex communication.

Light source 210 may be, for example, a light emitting diode (LED). The light source 210 may be configured to generate probe signals having one or more wavelengths of light. In some embodiments, the light source 210 may be configured to generate probe signals having at least two different wavelengths of light. In other embodiments, the light source 210 may also be configured to generate probe signals having at least three different wavelengths of light. In some embodiments, light source 210 may be a composite light source, formed from a plurality of individual light sources.

In some embodiments, the mode of operation of the light source 210 may be controlled remotely by terminal 120. For example, a user operating terminal 120 may send instructions over network 130 to operate light source 210 in certain modes. In some embodiments, the user operating terminal 120 may instruct that the light source 210 to emit probe signals having specific wavelengths of light. The mode of operation may also be controlled internally through processor 202 according to pre-determined instructions stored in memory 204 and executed by processor 202.

To detect the reflection or transmission of light through gastrointestinal fluids, the ingestible device 110 may be also equipped with a first optical sensor 212 and, in at least some embodiments, a second optical sensor 214. Optical sensors 212 and 214 can be, for example, photodiodes. In some embodiments, a plurality of optical sensors 212 and/or 214 may be provided.

The optical sensors 212, 214 may be configured to output a received signal in response to and corresponding to the detection of a light signal. The received signal may comprise, for example, analog voltages. The value of the analog voltage may correspond to an intensity of one or more preselected wavelengths of light. In some embodiments, the first or second optical sensors 212, 214 may be adapted to detect an intensity of red, green, and blue light, and to generate an analog voltage output corresponding to a red channel, green channel, and blue channel. The received signals can then be transmitted to processor 202.

Image sensor 216 can be a camera operable to capture images. The images captured by image sensor 216 may be transmitted to the remote terminal 120. The image sensor 216 may be prompted to capture an image, for example, by instructions sent from an operator of terminal 120. In some embodiments, the image sensor 216 may be automatically prompted by processor 202 to capture an image in response to a specific event, such as, e.g., the detection of a light signal within predetermined parameters.

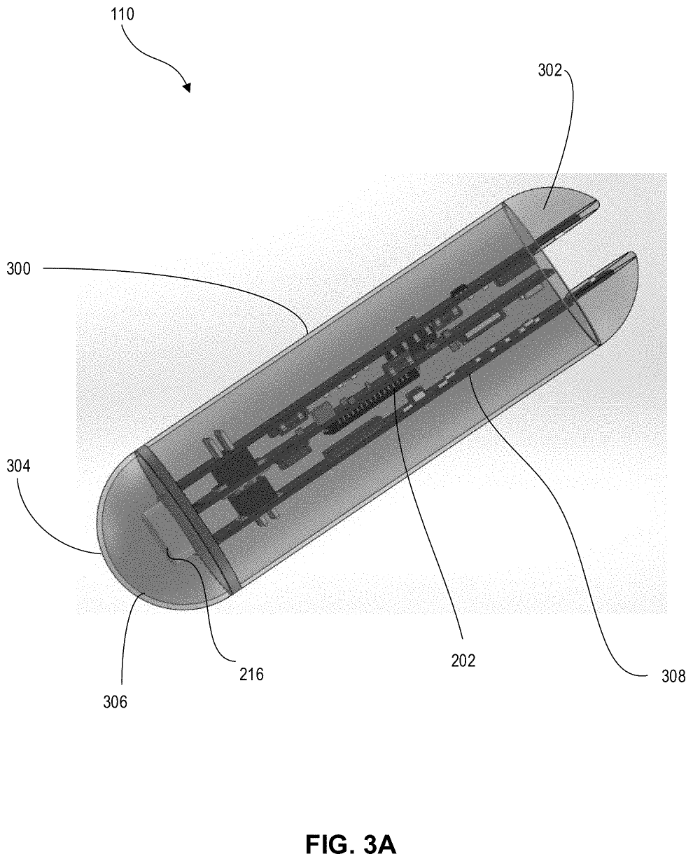

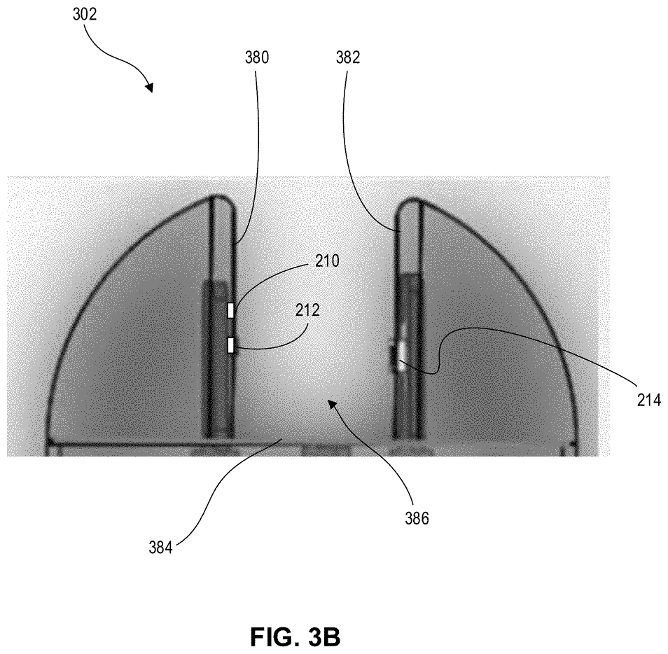

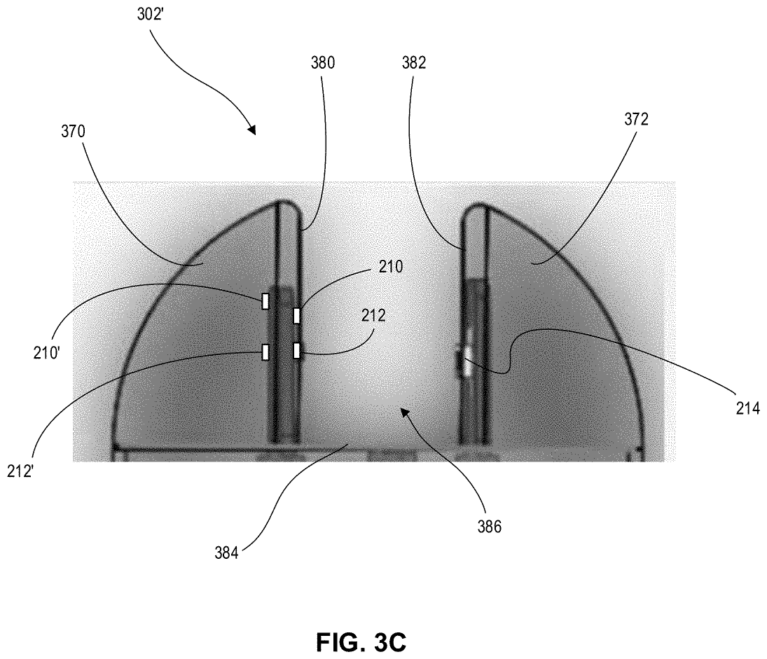

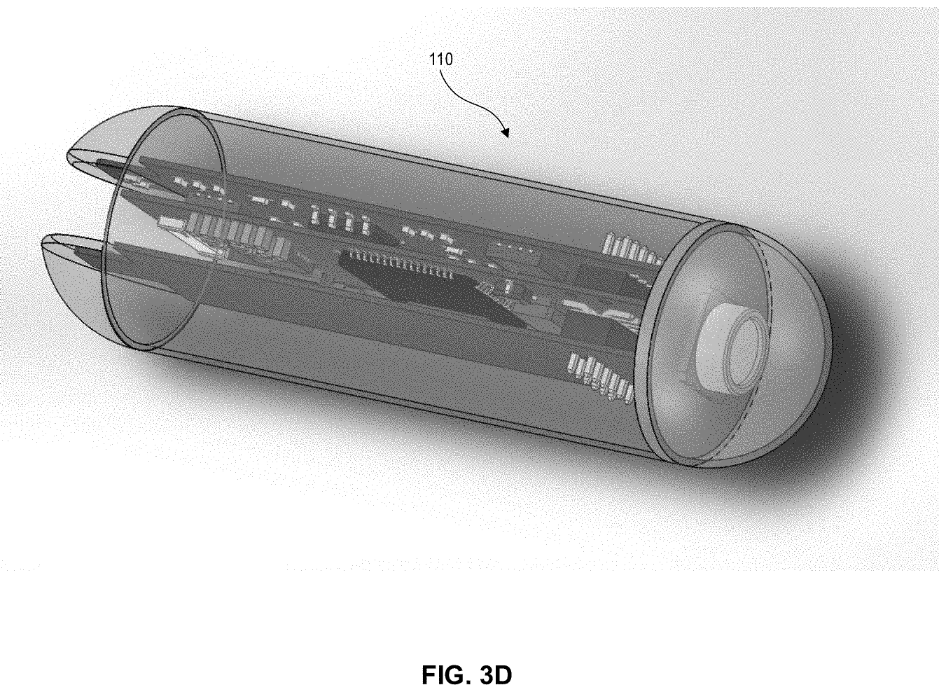

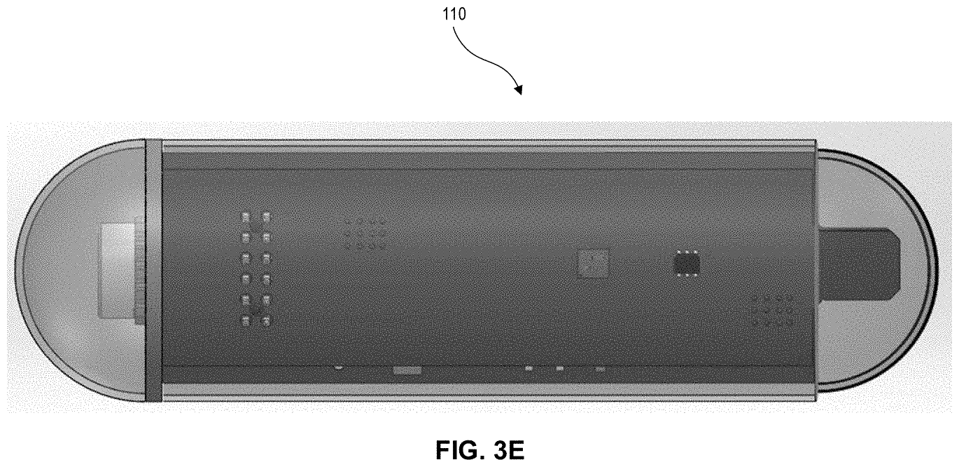

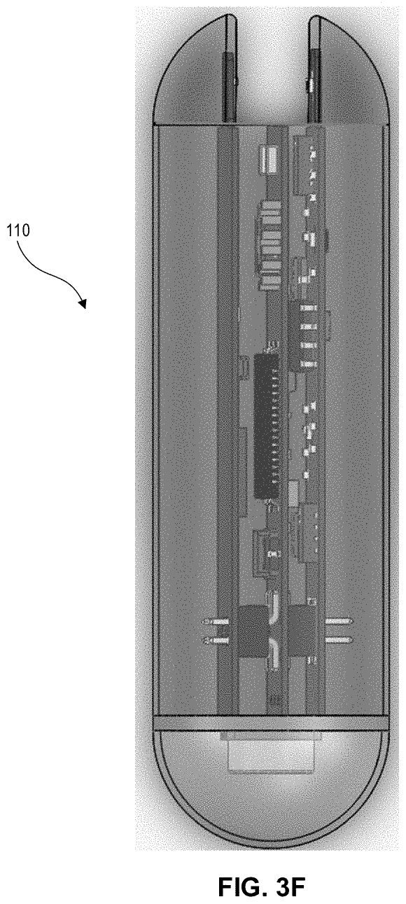

Referring now to FIG. 3A, there is shown a side perspective view of an example ingestible device 110 in accordance with some embodiments.

In the illustrated example, ingestible device 110 has an elongated ovoid or generally cylindrical body 300. The elongated body 300 may have at least a first end portion 302, and a second end portion 304, wherein the second end portion 304 may be distally opposed from the first end portion 302.

The first end portion 302 will be described in further detail below, with reference to FIG. 3B.

The second end portion 304 has a rounded or semi-spherical cap 306 that acts as a cover for image sensor 216, which is itself provided on the body 300. The cap 306 may be substantially transparent in order to allow images, or video, to be captured by an image sensor 216.

While the body 300 has been generally illustrated as cylindrically shaped, the body 300 is not limited to any one particular shape or dimension. Moreover, body 300 is only limited in size in so far as the ingestible device 110 should be adapted to be swallowed or ingested by a user or patient. The outer-shell of body 300 may be manufactured from a material that is suitable for human ingestion, but that will resist digestion and decomposition while passing through the gastrointestinal tract.

Housed within body 300 is electronic circuit 308. The electronic circuit 308 may include, for example, at least the processor 202, memory 204, communication module 206, and power module 208, as described herein.

Referring now to FIG. 3B, there is illustrated an enlarged side perspective view of the first end portion 302 of ingestible device 110.

The first end portion 302 generally has a recess 386 defined by a first sidewall 380, a second sidewall 382, and a base wall 384. The first sidewall 380 and the opposing second sidewall 382 are laterally spaced apart at least by the lateral span of the base wall 384.

In the illustrated example, at least one light source 210 is provided on or in first sidewall 380. A first optical sensor 212 is provided in sidewall 380, such that the first optical sensor 212 acts as a reflected light optical sensor (or “reflectance sensor”). A second optical sensor 214 is provided in sidewall 382, such that the second optical sensor 214 acts as a transmitted light optical sensor (or “transmittance sensor”).

In some embodiments, one or more light source 210 may be disposed on one or both sidewalls 380 and 382. Likewise, in some embodiments, one or more optical sensors 212 and 214 may be provided in one or both sidewalls 380 and 382, and operated in cooperation with light sources 210 to detect reflected light or transmitted light, as described further herein.

Recess 386 is provided and adapted to allow a fluid to pass through the recess. The fluid, inside of the GI tract, may comprise gastrointestinal fluids, which may in some cases have the target substance (e.g., blood) therein.

To measure the reflectance properties of the fluid in recess 386, the light source 210 may emit one or more probe light signal in the direction of the fluid. The reflection of the probe signal from the fluid can be detected using a reflectance sensor (e.g., optical sensor 212, located on the same sidewall as the light source 210).

To measure the transmission properties of the fluid in recess 386, the light source 210 may emit a probe light signal at the fluid. The transmission of the probe signal through the fluid may then be detected using a transmittance sensor (e.g., optical sensor 214, located on a sidewall opposite to the light source).

Referring now to FIG. 3C, there is illustrated an enlarged side perspective view of the first end portion 302′ of ingestible device 110 in accordance with another example embodiment.

The first end portion 302′ may be analogous to first end portion 302 of FIG. 3B except in that it may have one or more outwardly directed light sources 210′ and sensors 212′. In some cases, a recess in first end portion 302′ may be omitted. First end portion may have a substantially transparent cover 370. Optionally, cover 372 may also be substantially transparent.

In the illustrated example, at least one light source 210′ is provided on or in sidewall 380 and facing outwardly relative to a central axis of device 110 (and recess 386).

Likewise, an optical sensor 212′ is provided on or in sidewall 380, such that the optical sensor 212 acts as a reflectance sensor for light transmitted by light source 210′ and reflected from, e.g., intestinal walls or intraintestinal fluids.

In some embodiments, one or more light source 210′ may be disposed on or in one or both sidewalls 380 and 382. Likewise, in some embodiments, one or more optical sensors 212′ may be provided on or in one or both sidewalls 380 and 382, and operated in cooperation with light sources 210′ (or 210) to detect reflected light, as described further herein.

Referring now to FIG. 3D, there is illustrated a perspective view of the device 110 of FIG. 3A from the second end portion.

Referring now to FIG. 3E, there is illustrated a rotated side view of the device 110 of FIG. 3A, showing a face of the bottom of a circuit board.

Referring now to FIG. 3F, there is illustrated another rotated side view of the device 110 of FIG. 3A, showing the circuit boards in profile.

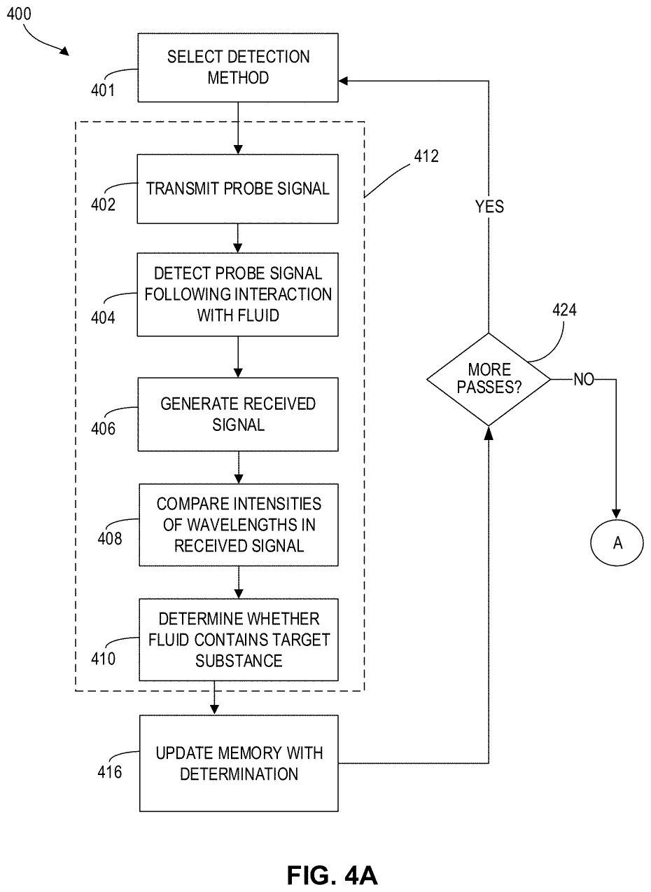

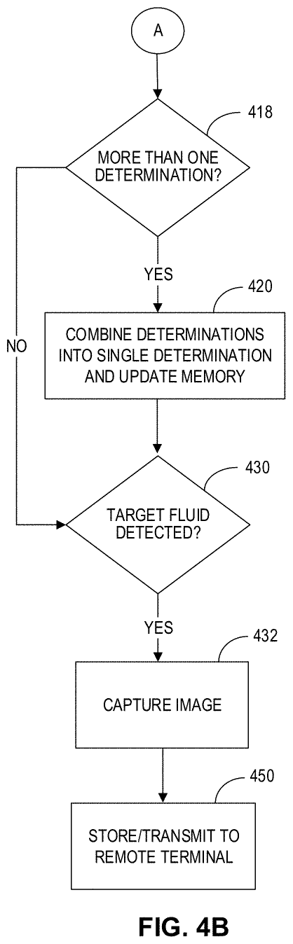

Referring now to FIG. 4A and FIG. 4B, there is illustrated an example process flow for a method of in situ detection of a composition of a fluid in a gastrointestinal tract in accordance with some embodiments. Method 400 may be carried out, for example, using ingestible device 110 of FIG. 1.

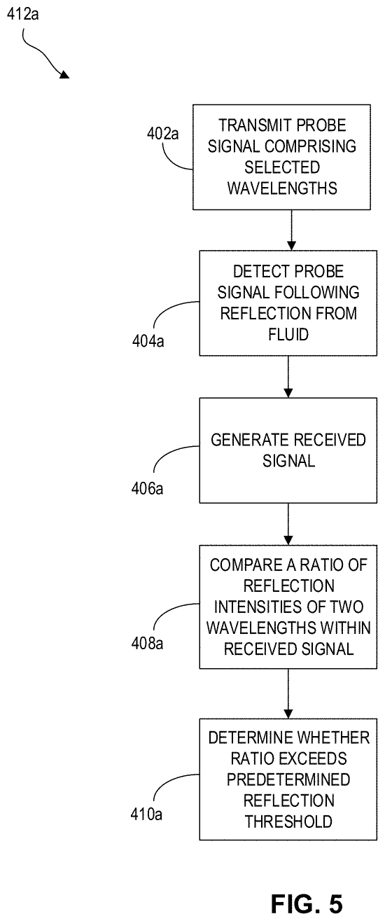

At 401, one or more detection method can be selected from one or more predetermined detection methods available to the wireless capsule, as described in further detail with reference to FIGS. 5 to 10. The one or more detection methods may be selected, for example, by the processor of the wireless capsule, or at remote terminal 120, in which case they may be communicated to the processor. In the event that multiple detection methods are selected, a multi-pass detection may be performed as described herein. Although illustrated as multiple sequential passes to ease understanding, in some cases, the acts of multiple detection methods may be performed contemporaneously (e.g., a common probe signal may be used to determine reflected light and transmitted light).

At 402, a probe signal may be emitted from light source 210 at a fluid contained within recess 386. The content of the probe signal (i.e., the number, intensity and duration or pattern of selected wavelengths) may be determined based on the method selected at 401 and as described further herein. For example, in some embodiments, the probe signal can comprise at least two wavelengths of light with respective intensities. In other embodiments, the probe signal can comprise at least three wavelengths of light with respective intensities.

At 404, following an interaction of the probe signal with the fluid, the probe signal may be detected by an optical sensor. When the interaction of the probe signal with the fluid is a reflection of light from the fluid, then the probe signal may be detected by a reflectance sensor, such as optical sensor 212. When the interaction of the probe signal with the fluid is a transmission of light through the fluid, then the probe signal may be detected by a transmittance sensor, such as optical sensor 214.

At 406, the optical sensor 212 or 214 which detects the probe signal can generate a received signal. The received signal can indicate the intensity and duration of the one or more wavelengths of light in the detected probe signal.

At 408, the intensities of the wavelengths in the received signal can be determined and, in some cases, compared according to one or more formula as described further herein. This determination and comparison can be performed by the processor 202.

At 410, based on the results of the comparison at 408, a determination is made by the processor as to whether the fluid contains a target substance. The target substance may be, for example, hemoglobin (i.e., in a blood solution). This may indicate an abnormality in the gastrointestinal tract, such as chronic or acute bleeding.

At 416, memory 204 of ingestible device 110 can be updated to reflect the determination made at 410. For example, the processor 202 may communicate the determination of 410 to the memory 204 for storage. In some cases, the processor may also transmit the determination and/or images taken in conjunction with the reception of the light signals, to the remote terminal.

At 424, the processor 202 can determine whether there are additional passes to be made, e.g., additional detection methods to be applied, in which case the processor may return to 401 and begin an additional pass. As noted above, although illustrated as sequential passes to ease understanding, in some embodiments, the acts of multiple detection methods may be performed contemporaneously (e.g., a common probe signal may be used to determine reflected light and transmitted light).

If there are no further passes to be made, the processor 202 may proceed to 418.

At 418, the processor 202 can assess whether more than one detection method has been used (e.g., whether there is more than one determination stored in memory 204 from previous iterations of steps 401 to 410). When there is more than one determination, the processor 202 can combine these determinations into a single combined determination at 420. In some embodiments, the determinations can be combined together by averaging the separate determinations (i.e., a weighted or non-weighted averaging of the determinations). In some embodiments, previous determinations can be discarded in favor of newer determinations. In some embodiments, all determinations can be preserved separately in memory 204, in which case 420 may be omitted.

Based on the combined determination, or individual determinations, the processor may at 430 determine whether a target substance has been detected in the fluid under examination. In response to detection of the target substance, the processor 202 may cause the image sensor 216 to capture one or more images at 432 that can be correlated to the target substance determination. In some embodiments, images may be captured periodically or continuously instead, regardless of the determination.

The image sensor 216 may be used capture an image of a region, or area, of the gastrointestinal tract in close proximity to the in situ detection being conducted. Thus, for example, if the processor 202 determines at 410 that the target substance (e.g., blood) is present in the fluid under examination, an image of the corresponding portion of the GI tract can be captured.

At 450, the combined determination from 420, or the individual determinations, can be correlated to captured images based on, e.g., a timestamp, and stored locally for later retrieval, or may be transmitted to remote terminal 120. The determinations and captured images can be used by a clinician, for example to aid the clinician in identifying the portion of the GI tract in which the target substance is detected.

Referring now to FIGS. 5 to 10, several example detection methods for in situ detection of a target substance are further described. Each of the example detection methods may be used to carry out, e.g., acts 402 to 410 of method 400.

Referring first to FIG. 5, there is illustrated a process flow diagram for a method 412a for in situ detection of a target substance in accordance with some embodiments. Method 412a may be used to analyze the reflection properties of a fluid, and a target substance in the fluid, in recess 386 of ingestible device 110.

At 402a, a probe signal may be emitted at the fluid using light source 210. The probe signal may comprise at least two selected wavelengths of light. The at least two selected wavelengths of light may be predetermined based on experimental data. For example, in some embodiments, the probe signal may comprise a first selected wavelength at substantially 700 nm and a second selected wavelength at substantially 630 nm, which may be indicative of blood in solution. In other embodiments, the probe signal may comprise a first selected wavelength at substantially 480 nm and a second selected wavelength at substantially 530 nm, which also may be indicative of blood in solution. The wavelength pairs are predetermined based on experimental data showing a high degree of selectivity for blood as a target substance.

At 404a, a reflectance sensor, such as optical sensor 212 may detect the probe signal following its reflection from the fluid. As described herein, in order to detect the reflection of the probe signal, the reflectance sensor may be positioned on a same sidewall as the light source 210.

At 406a, the reflectance sensor may generate a received signal indicative of the intensity of the selected wavelengths of light in the detected probe signal. The received signal may be transmitted to the processor 202.

At 408a, the system may compare a ratio of reflection intensities of the at least two selected wavelengths within the received signal. The ratio of reflection intensities may be calculated according to =Equation (1):

R=I1/I2−C (1)

where R is the reflection intensity ratio, I1 is the reflection intensity of the first selected wavelength, I2 is the reflection intensity of the second selected wavelength and C is a cut-off value (which may be set to zero in some cases).

At 410a, a determination can be made as to whether the fluid contains a target substance (i.e., hemoglobin, or is otherwise a blood solution). The determination may be made by considering whether the reflection intensity ratio exceeds a predetermined reflection threshold.

The value for the predetermined reflection threshold can be based on the constituent wavelengths of the probe signal at 402a. For example, for probe signals generated with the selected wavelengths at substantially 700 nm and 630 nm, the value of the reflection threshold may be substantially within the range of 1.2 to 1.3. For probe signals with the selected wavelengths at substantially 480 nm and 530 nm, the reflection threshold may be substantially within the range of 0.9 to 1.0.

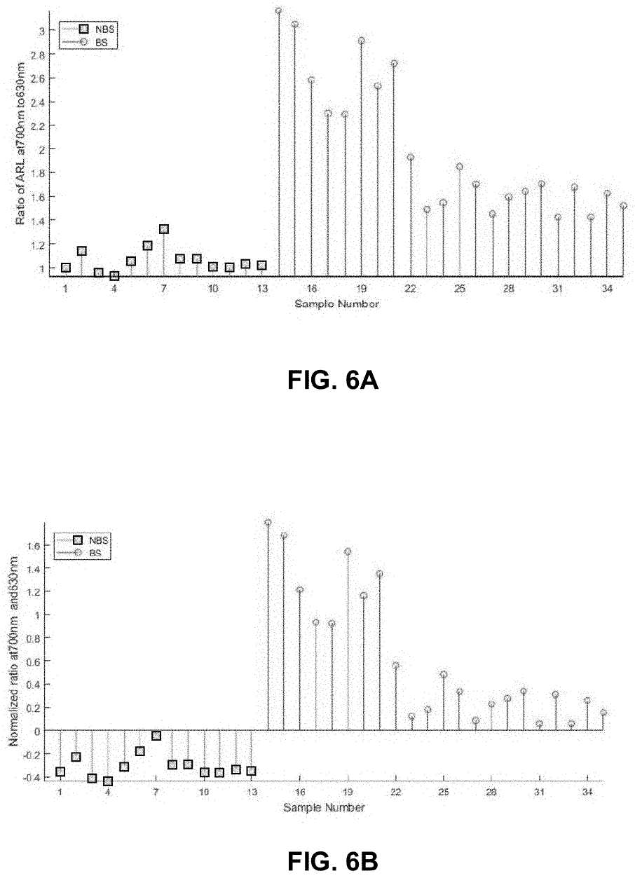

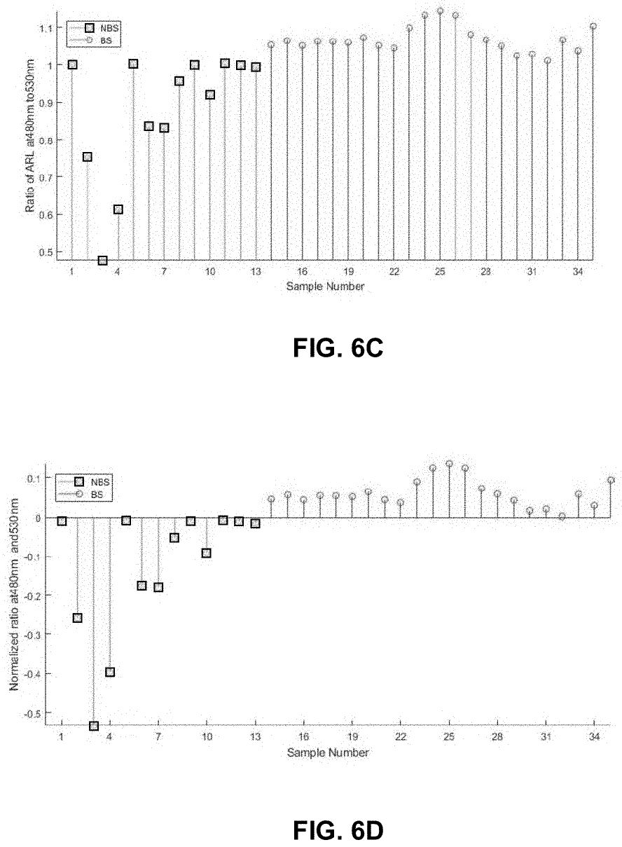

FIG. 6A to 6D are plots illustrating experimental data generated according to the method of FIG. 5.

FIG. 6A shows a plot of the reflection intensity ratios from experimental analysis of blood and non-blood samples, which were subjected to probe signals having wavelengths at substantially 700 nm and 630 nm. The initial thirteen samples, as shown on the X-axis of plot (samples 1 to 13), comprise an array of non-blood solutions. The subsequent twenty-two samples (samples 14 to 35), comprise blood solutions with varying levels of hemoglobin. In the plot of FIG. 6A, the cut-off value C is set to zero. Bovine, equine and swine blood samples were used in experiments for the blood solutions as a proxy for human blood.

FIG. 6B shows the plot of FIG. 6A in which the cut-off value is set to 1.3664. As shown in FIG. 6B, selection of a cut-off value serves to easily distinguish between non-blood samples (in which the reflectance intensity ratio is negative) and blood samples (which have a positive reflectance intensity ratio). In particular, the reflection ratios for non-blood samples (samples 1 to 13), are consistently located below the X-axis, which serves as a cut-off indicator line. Comparatively, the reflection ratios for blood samples (samples 14 to 35), are consistently located above the cut-off indicator line.

TABLE 1

| | Actual Blood Sample | Actual Non-Blood Sample |
|---|---|---|
| Detected as | 22 | 0 |
| Blood Sample | | |
| Detected as | 0 | 13 |
| Non-Blood Sample | | |

FIG. 6C shows a plot of the reflection intensity ratios from experimental analysis of blood and non-blood samples, which were subjected to probe signals having wavelengths at substantially 480 nm and 530 nm. The initial thirteen samples, as shown on the X-axis of plot (samples 1 to 13), comprise an array of non-blood solutions. The subsequent twenty-two samples (samples 14 to 35), comprise blood solutions with varying levels of hemoglobin. In the plot of FIG. 6A, the cut-off value C is set to zero.

FIG. 6D shows the plot of FIG. 6C in which the cut-off value is set to 1.0095. As shown by FIG. 6D, selection of a cut-off value serves to easily distinguish between non-blood samples (in which the reflectance intensity ratio is negative) and blood samples (which have a positive reflectance intensity ratio). In particular, the reflection ratios for non-blood samples (samples 1 to 13), are consistently located below the X-axis, which serves as a cut-off indicator line. Comparatively, the reflection ratios for blood samples (samples 14 to 35), are consistently located above the cut-off indicator line.

TABLE 2

| | Actual Blood Sample | Actual Non-Blood Sample |
|---|---|---|
| Detected as | 22 | 0 |
| Blood Sample | | |
| Detected as | 0 | 13 |
| Non-Blood Sample | | |

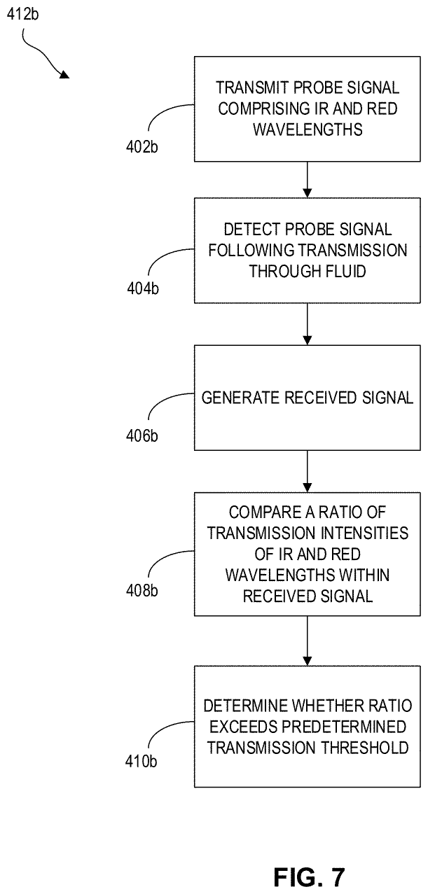

Referring now to FIG. 7, there is illustrated a process flow diagram for a method 412b for in situ detection of a target substance in accordance with some embodiments. Method 412b may be used to analyze the transmission properties of a fluid, and a target substance in the fluid, in recess 386 of ingestible device 110.

At 402b, a probe signal may be emitted at the fluid using light source 210. The probe signal may comprise a red wavelength light (e.g., substantially around 660 nm) and an infrared (IR) wavelength light (e.g., substantially around 880 nm).

At 404b, a transmittance sensor, such as optical sensor 214, may detect the probe signal following its transmission through the fluid. As described herein, in order to detect the transmission of the probe signal, the transmittance sensor may be positioned on a sidewall opposite to the sidewall where the light source 210 is disposed.

At 406b, the transmittance sensors may generate a received signal indicative of the intensity of each of the wavelengths in the detected probe signal. The received signal may be transmitted to the processor 202.

At 408b, the system may compare a ratio of transmission intensities of the two wavelengths within the received signal. The ratio of transmission intensities R_{RIR} may be calculated according to Equation (2), wherein I_{red} denotes a received intensity of the red wavelength and I_{IR} denotes a received intensity of the infrared wavelength, and C is a cut-off value initially set to zero:

R_{RIR}=I_{Red}/I_{IR}−C (2)

At 410b, a determination can be made as to whether the fluid contains a target substance (i.e., contains hemoglobin, or is otherwise a blood solution). The determination can be made by considering whether the calculated ratio of Equation (5) exceeds a predetermined transmission threshold. The predetermined transmission threshold may have a value of substantially 101, within about 10%, preferably within about 5% and still more preferably within about 1%.

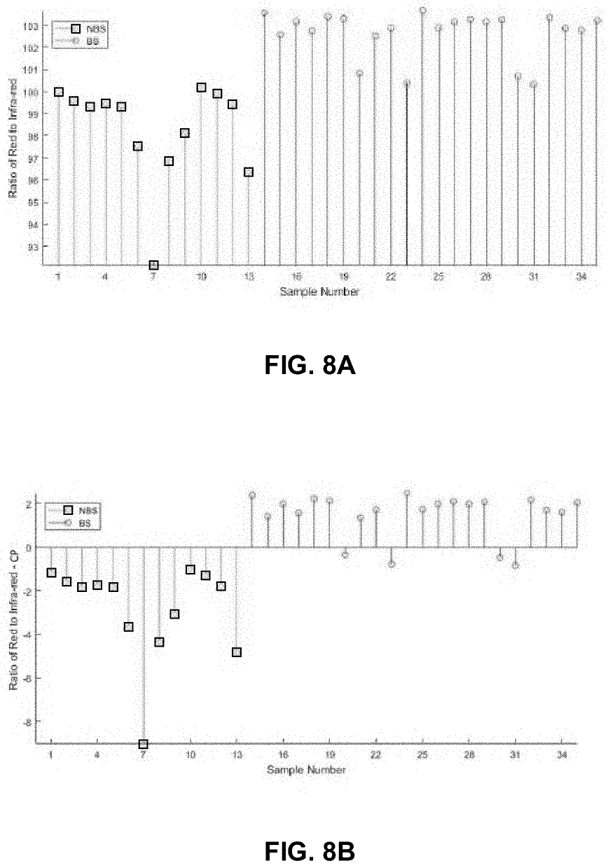

FIGS. 8A and 8B are plots illustrating experimental data generated according to the method of FIG. 7.

FIG. 8A shows a plot of transmission ratios from experimental analysis of blood and non-blood samples, which were subjected to probe signals having wavelengths at substantially red and IR wavelengths. The first thirteen samples, as shown on the X-axis of plot (samples 1 to 13), comprise an array of non-blood solutions. The later twenty-two samples (samples 14 to 35), comprise blood solutions with varying levels of hemoglobin.

FIG. 8B shows the plot of FIG. 8A in which a cut-off value is set to 101.1743. As shown in FIG. 6B, the selection of cut-off value serves to easily distinguish between non-blood and blood samples. In particular, the transmission ratios for non-blood samples (samples 1 to 13) are consistently located below the normalized cut-off line, and do not otherwise satisfy the condition of Equation (6). Comparatively, the transmission ratios for the blood samples (samples 14 to 35) are generally located above the normalized cut-off line (with the exception of samples 20, 23, 30, and 31), and generally satisfy the condition of Equation (6):

TABLE 3

| | Actual Blood Sample | Actual Non-Blood Sample |
|---|---|---|
| Detected as | 18 | 0 |
| Blood Sample | | |
| Detected as | 4 | 13 |
| Non-Blood Sample | | |

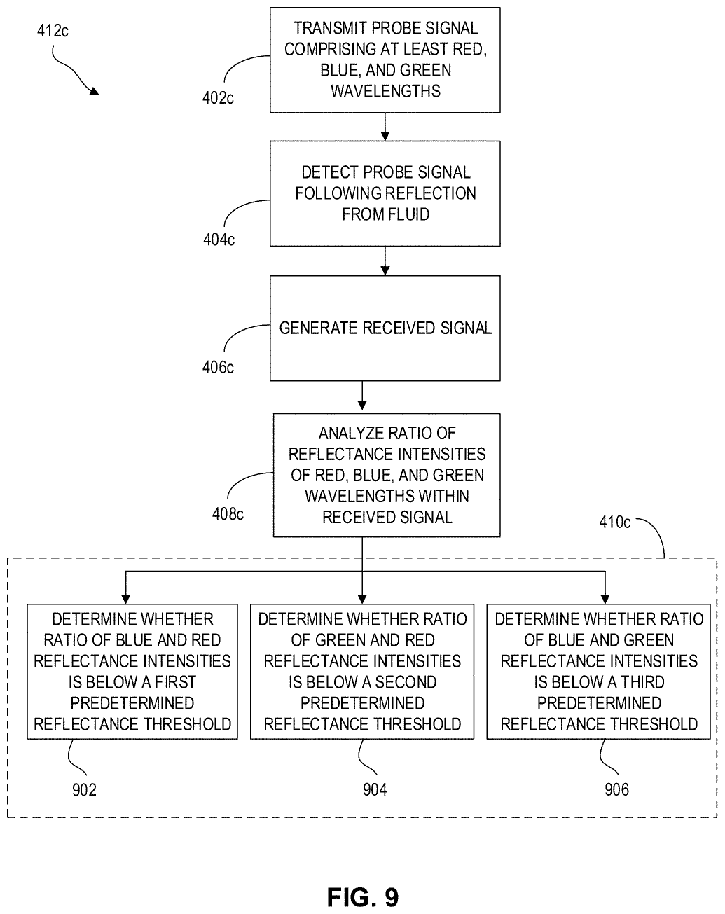

Referring first to FIG. 9, there is illustrated a process flow diagram for a method 412c for in situ detection of a target substance in accordance with some embodiments. Method 412c may be used to analyze the reflection properties of a fluid in response red, blue and green light wavelengths from a target substance in the fluid, in recess 386 of ingestible device 110.

At 402c, a probe signal may be emitted at the fluid using light source 210. The probe signal may comprise red, blue and green wavelengths of light. In some embodiments, the probe signal may simply comprise a white light.

At 404c a reflectance sensor, such as optical sensor 212 may detect the probe signal followings its reflection from the fluid. As described herein, in order to detect the reflection of the probe signal, the reflectance sensor may be positioned on the same sidewall as the light source 210.

At 406c, the reflectance sensor may generate a received signal indicative of the intensity of the red, green and blue wavelengths in the detected probe signal. The received signal may comprise, for example, a plurality of voltages, wherein each voltage corresponds to a reflection intensity of each of the red, blue, and green wavelengths, respectively. The received signal may be transmitted to the processor 202.

At 408c, the system may analyze the reflection intensities of each of the red, blue, and green wavelengths. In one embodiment, the analysis at 408c may comprise: (1) normalizing the respective intensities of each wavelength against the reflection intensities of the same wavelengths in water, and (2) subtracting the mean value of each reflection intensity from the respective intensity value.

At 410c, a determination can be made as to whether the fluid contains a target substance (i.e., contains hemoglobin, or is otherwise a blood solution). The determination may be made according to any one or more of Equations (3), (4), or (5):

Inb−x*Inr≤0 (3)

Ing−y*Inr≤0 (4)

Inb−z*Ing≤0 (5)

where Inb is the normalized blue wavelength reflection intensity, Ing is the normalized green wavelength reflection intensity, Inr is the normalized red wavelength reflection intensity, and where x, y and z are comparison factors. In at least one embodiment, x is substantially 0.0814. In at least one embodiment, y is substantially 0.1083. In at least one embodiment, z is substantially 0.6911.

Equations (3), (4), and (5) correspond to steps 902, 904, and 906, respectively. In some embodiments, the determinations of Equations (3), (4), and (5) may be combined to generate a final determination (i.e., using a weighted or non-weighted averaging). This may improve an overall accuracy of the generated determinations.

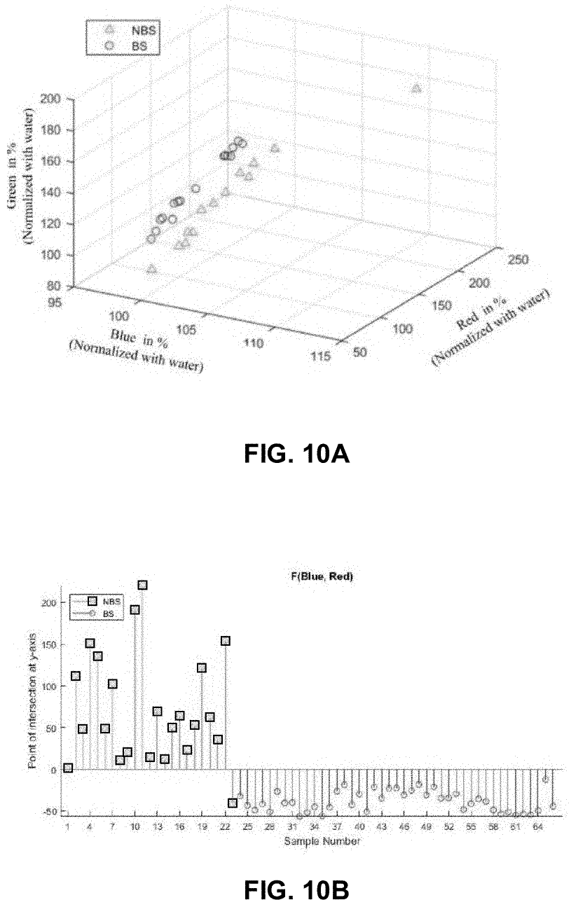

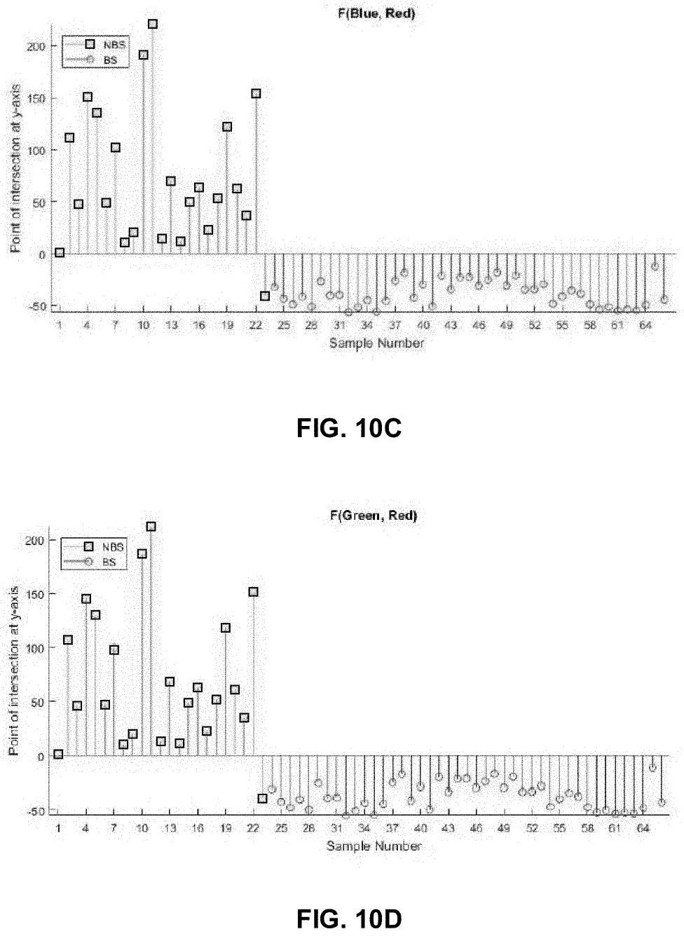

FIGS. 10A to 10C are plots illustrating experimental data generated according to the method of FIG. 9.

FIG. 10A shows a plot of the reflection intensities of red, blue, and green wavelengths from a number of blood and non-blood samples. The reflection intensity values shown in FIG. 10A are normalized against the reflection intensities of the same wavelengths in water.

As shown by FIG. 10A, the blood and non-blood samples occupy separate regions of the plot. The samples are accordingly separable using at least a line or plane of separation (e.g., a line or two-dimensional plane in accordance with any one of Equations (3) to (5).

FIG. 10B shows a normalized plot after applying Equation (3). As shown, the non-blood samples (samples 1 to 23) are generally located above the normalized line, and do not otherwise satisfy the condition of Equation (3). Comparatively, blood samples (samples 24 to 66) are consistently located below the normalized cut-off line, and accordingly satisfy the condition of Equation (3).

FIGS. 10C and 10D show equivalent plots using Equations (4) and (5), respectively.

TABLE 4

| SL | | | Optimum | Accuracy of |
|---|---|---|---|---|
| No | x | y | value of m | Separation |
| 1 | Blue | Red | 0.0814 | 98.48% |
| 2 | Green | Red | 0.1083 | 98.48% |
| 3 | Blue | Green | 0.6911 | 95.45% |

As described herein, any one or more of the detection methods 410a to 410c can be incorporated into the detection method 400 of FIG. 4.

The present invention has been described here by way of example only, while numerous specific details are set forth herein in order to provide a thorough understanding of the exemplary embodiments described herein. However, it will be understood by those of ordinary skill in the art these embodiments may, in some cases, be practiced without these specific details. In other instances, well-known methods, procedures and components have not been described in detail so as not to obscure the description of the embodiments. Various modifications and variations may be made to these exemplary embodiments without departing from the spirit and scope of the invention, which is limited only by the appended claims.

1. A method of in situ detection of a composition of a fluid within a gastrointestinal tract using an ingestible device, the method comprising:
transmitting, using at least one light transmission source of the ingestible device, at least one probe signal from the at least one light transmission source, the at least one probe signal comprising at least two wavelengths of light with respective intensities;
detecting, using at least one optical sensor of the ingestible device, the at least one probe signal following at least one interaction of the at least one probe signal with the fluid to generate a received signal;
comparing the respective intensities of the at least two wavelengths of light within the received signal;
based on the comparing, determining whether the fluid contains a target substance; and
updating a memory of the ingestible device with the determination.

2. The method of claim 1, wherein the interaction of the at least one probe signal with the fluid comprises a reflection from the fluid, and wherein the comparing comprises comparing a ratio of respective reflection intensities of the at least two wavelengths of light from the fluid.

3. The method of claim 2, wherein the first wavelength is substantially about 700 nm and the second wavelength is substantially about 630 nm.

4. The method of claim 2, wherein the first wavelength is substantially about 480 nm and the second wavelength is substantially about 530 nm.

5. The method of claim 1, wherein the determining comprises determining whether the ratio exceeds a predetermined reflectance threshold.

6. The method of claim 1, wherein the interaction of the at least one probe signal with the fluid comprises a transmission through the fluid, and wherein the comparing comprises comparing respective transmission intensities of the at least two wavelengths of light through the fluid with a predetermined transmission threshold.

7. The method of claim 6, wherein the first wavelength corresponds to red light and the second wavelength corresponds to infrared light.

8. The method of claim 7, wherein the first wavelength is substantially about 660 nm and the second wavelength is substantially about 880 nm.

9. The method of claim 6, wherein the predetermined transmission threshold is substantially 101.

10. The method of claim 1, wherein the interaction of the at least one probe signal with the fluid comprises a reflection from the fluid, wherein the at least one probe signal comprises at least three wavelengths of light, and wherein the comparing comprises comparing respective reflectance intensities of the at least three wavelengths of light from the fluid.

11. The method of claim 10, wherein the first wavelength corresponds to red light, the second wavelength corresponds to green light and the third wavelength corresponds to blue light.

12. The method of claim 11, wherein the comparing comprises comparing reflectance intensities of each of the at least three wavelengths of light to one or more predetermined reflectance thresholds.

13. The method of claim 12, wherein a first predetermined reflectance threshold of the one or more predetermined reflectance thresholds is determined according to the formula:
Inb−x*Inr
wherein Inb is a reflectance intensity of the third wavelength, Inr is a reflectance intensity of the first wavelength, and x is a first comparison factor.

14. The method of claim 13, where x is substantially 8%.

15. The method of claim 12, wherein a second predetermined reflectance threshold of the one or more predetermined reflectance thresholds is determined according to the formula:
Inb−y*Inr
wherein Ing is a reflectance intensity of the second wavelength, Inr is a reflectance intensity of the first wavelength, and y is a second comparison factor.

16. The method of claim 15, where y is substantially 10%.

17. The method of claim 12, wherein a third predetermined reflectance threshold of the one or more predetermined reflectance thresholds is determined according to the formula:
Inb−z*Ing
wherein Inb is a reflectance intensity of the third wavelength, Ing is a reflectance intensity of the second wavelength, and z is a third comparison factor.

18. The method of claim 17, where z is substantially 69%.

19. A non-transitory computer readable medium storing one or more computer-executable instructions, which when executed by a processor, causes the processor to perform the method of claim 1.

20. An ingestible device comprising:
an electronic circuit, the electronic circuit comprising a processor, a memory and an energy storage element;
a device body with a first end and a second end opposed along a longitudinal axis;
a first end portion of the device body positioned at the first end of the device body along the longitudinal axis, the first end portion having a recess therein, the recess defined by a first sidewall, a second sidewall and a base wall, the first and second sidewalls laterally spaced apart;
at least one light transmission source coupled to the electronic circuit and positioned to emit light generally perpendicularly to the first sidewall; and
at least one optical sensor coupled to the electronic circuit and positioned in at least one of the first sidewall and the second sidewall.
